(19) United States
(12) Patent Application Publication
(10) Pub. No.: US 2008/0058733 A1
(43) Pub. Date: Mar. 6, 2008

(54) ANTIBIOTIC(S)-POLYMER COMBINATION

(75) Inventors: Sebastian Vogt, Jena (DE); Matthias Schnabelrauch, Marburg (DE); Klaus Dieter Kuhn, Marburg (DE)

Correspondence Address: NORRIS, MCLAUGHLIN & MARCUS, PA, 875 THIRD AVENUE, 18TH FLOOR, NEW YORK, NY, 10022, US

(73) Assignee: HERAEUS KULZER GMBH & CO. KG, Hanau (DE)

(21) Appl. No.: 11/931,895

(22) Filed: Oct. 31, 2007

Related U.S. Application Data
(63) Continuation of application No. 10/100,865, filed on Mar. 19, 2002.

(30) Foreign Application Priority Data
Mar. 22, 2001 (DE) 10114247.1

(51) Int. Cl. A61F 2/00; A61B 17/56; A61K 31/08; A61K 9/14; A61M 5/32; A61K 35/00; A61B 17/58

(52) U.S. Cl. 604/265; 424/423; 424/489; 424/780; 427/2.24; 514/722; 606/69; 606/72; 606/73

(57) ABSTRACT

The present invention relates to an antibiotic(s)-polymer combination, which under physiological conditions guarantees the continuous release of antibiotics over a period of several days and can be used in human and veterinary medicine. The invented antibiotic(s)-polymer combination is wherein in a homogeneous polymer mixture, consisting of one or more hydrophobic polymers from the groups of poly(methacrylic acid esters), the poly(acrylic acid esters) and the poly(methacrylic acid ester-co-acrylic acid esters) and one or more hydrophilic polymers from the group of polyethers, one or more slightly water-soluble antibiotics from the groups of aminoglycoside antibiotics, the lincosamide antibiotics, the tetracycline antibiotics and quinolone antibiotics, possibly an easily water-soluble antibiotic from the groups of aminoglycoside antibiotics, the lincosamide antibiotics and the tetracycline antibiotics, and possibly one or more organic adjuvants are suspended, and that this suspension forms a composite.

The present invention relates to an antibiotic(s)-polymer combination, which under physiological conditions guarantees the continuous release antibiotics over a period of several days and can be used in human and veterinary medicine.

In human and veterinary medicine, medicinal products made from polymers are used in the form of drainages, catheters, cover foils and nets as temporary or permanent implants for secretion removal, rinsing, covers and fixation. The problem with this is that micro-organisms can migrate into the organism especially in the case of drainages and catheters along these plastic tubes and can thus cause local infections, which if untreated can be spread further in the organism. Similar problems occur with the usage of fixation devices externally. There, microorganisms can penetrate into the organism similarly along the pins. Also in the case of dental implants infection problems on the implant surface are known. This leads to the necessity that for medical applications of these implants, infection prophylaxis or infection control must occur. Suppressing such infections can basically take place systemically or locally with suitable antibiotics. The systemic application of antibiotics is associated with a number of problems. In order to be able to obtain antimicrobially effective antibiotic concentrations systemically, relatively high antibiotics dosages are required. This can lead to undesirable damage, in particular for antibiotics of the aminoglycoside type and for antibiotics of the tetracycline type, due to their nephrotoxicity and/or ototoxicity. Thus, suppressing an infection through the local application of antibiotics is more advisable because effective local antibiotics concentrations can be reached while avoiding high systemic antibiotics concentrations.

The manufacture and usage of antibiotic polymer composites has been the object of intensive research for years, leading to a number of patents. For example Shepherd and Gould revealed a coating for catheters with hydrophilic polymethacrylates and polyacrylates, into which an antibiotic that is not described in detail is introduced for the treatment of infections (T. H. Shepherd, F. E. Gould: Catheter, Mar. 3, 1971, U.S. Pat. No. 3,566,874). Also disclosed by Shepherd and Gould is a retard system, described in the 1970s, on the basis of hydrophilic hydroxyalkylacrylates and hydroxymethacrylates, which are polymerized into antibiotically equipped molded bodies (T. H. Shepherd, F. E Gould: Dry hydrophilic acrylate or methacrylate polymer prolonged release drug implants, Dec. 31, 1974, U.S. Pat. No. 3,857,932). Klemm describes synthetic resin particles composed of polymethacrylate and polyacrylate for the treatment of osteomyelitis (K. Klemm: surgical synthetic-resin material and method of treating osteomyelitis, May 13, 1975, U.S. Pat. No. 3,882,858). These synthetic resin particles are impregnated with gentamycin or another antibiotic. Gross et al. reveals an advanced proposal for the production of bone cement that contains gentamicin (A. Gross, R. Schaefer, S. Reiss: Bone cement compositions containing gentamicin, Nov. 22, 1977, U.S. Pat. No. 4,059,684). Here salts that are easily dissolved in water, such as sodium chloride, potassium chloride, sodium bromide and potassium bromide, are added as adjuvants to a mixture consisting of pulverized copolymers of methyl-methacrylate and methylacrylate, methyl-methacrylate, gentamicin hydrochloride and/or gentamycin sulfate. This mixture was polymerized through peroxides. Upon introduction of the bone cement into a physiological environment, these salts are easily dissolved in water dissolve and leave cavities behind. Batich et al. described a new release system on a copolymer basis, which was synthesized while using weak-acid monomers and which swells beyond a pH value of 8.5 and thus is supposed to enable the release of enclosed pharmaceutical active ingredients (C. D. Batich, M. S. Cohen, K. Forster: Compositions and devices for controlled release of active ingredients, Oct. 10, 1996, U.S. Pat. No. 5,554,147).

The antimicrobial coating of medicinal products with antibiotic polymer systems was the object of a series of additional experiments. E.g. Conway et al. developed a polymer matrix made of silicone, in which water-soluble active ingredients on a nitrofuran basis were encapsulated in a thinly dispersed manner (A. J. Conway, P. J. Conway, R. D. Fryar Jr.: Sustained release bactericidal cannula, Nov. 16, 1993, U.S. Pat. No. 5,261,896). The usage of a matrix-forming polymer from the polyurethane, silicone and bio-degradable polymer groups, in which a mixture of silver salt and chlorhexidine has been suspended, was disclosed for the production of infection-resistant medicinal products (C. L. Fox Jr., S. M. Modak, L. A. Sampath: Infection-resistant compositions, medical devices and surfaces and methods for preparing and using same, May 28, 1991, U.S. Pat. No. 5,019,096). Solomon, Byron and Parke suggested similar anti-infective systems on the basis of polyurethane and chlorhexidine dispersed in it (D. D. Solomon, M. P. Byron: Anti-infective and antithrombogenic medical articles and method for their preparation, Sep. 19, 1995, U.S. Pat. No. 5,451,424; D. D. Solomon, M. P. Parke: Anti-infective and antithrombogenic medical articles and method for their preparation, Jan. 13, 1998, U.S. Pat. No. 5,707,366; D. D. Solomon, M. P. Parke: Anti-infective and antithrombogenic medical articles and method for their preparation, Jan. 13, 1998, U.S. Pat. No. 5,165,952). These systems were able to be processed from molten mass into molded bodies through an extrusion process. An antibiotic composition, which is composed of oligodynamically acting metals and polymers, was also revealed (D. Laurin, J. Stupar: Antimicrobial compositions, Jul. 29, 1984, U.S. Pat. No. 4,603,152). Acrylonitrile-butadiene-styrene copolymers, polyvinylchloride, polyester, polyurethane, styrene block copolymers and rubber, in which oligodynamically acting metals have been introduced for infection suppression purposes, are suggested as polymers. Elastomers can also be antibiotically outfitted. Allen for example created elastomer combinations of active substances by adding and incorporating active ingredients into rubber master batches (D. L. Allen: Elastomeric composition containing therapeutic agents and articles manufactured therefrom, May 28, 1991, U.S. Pat. No. 5,019,378). The master batches were composed of rubber, mica and titanium dioxide. An antibiotic coating consisting of a mixture of rifampin and minocycline, which were dispersed in a polymer, is suggested by Raad and Darouiche (I. I. Raad, R. O. Darouiche: Antibacterial coated medical implants, Jun. 8, 1993, U.S. Pat. No. 5,217,493). The polymer material, however, is not characterized in more detail there. De Leon et al. disclose a method for the antibiotic coating of implants on which the surface, which is supposed to be coated, is first covered with silicone oil (J. De Leon, T. H. Ferguson, D. S. Skinner Jr.: Method of making antimicrobial coated implants, Mar. 28, 1990, U.S. Pat. No. 4,952,419). In a second step, the pulverized active ingredient is applied onto the silicone oil layer. Oxytetracycline was used as the active ingredient. A similar coating on the basis of silicone oil and poly(methacrylic acid ester) was described by Takigawa, which was prepared from a solution of silicone oil and poly(methacrylic acid ester) in terpentine oil, N-decane, tetrachloromethane, butane-2-one, 1,4-dioxane, ethoxyethanol and toluene (B. Takigawa: Coating solution containing silicone oil and polymethacrylate, Feb. 24, 1998, U.S. Pat. No. 5,721,301). Mustacich et al. describe an antimicrobial polymer combination, where fatty acids and fatty acid salts are introduced into polymers for medical usage as biocide reagents (R. V. Mustacich, D. S. Lucas, R. L. Stone: Antimicrobial polymer compositions, Oct. 30, 1984, U.S. Pat. No. 4,479,795).

An interesting coating composition was disclosed by Whitbourne and Mangan, where the quaternary ammonium compounds are incorporated into a water-insoluble polymer, such as cellulose ester, as antimicrobial reagents (R. J. Whitbourne, M. A. Mangan: Coating compositions comprising pharmaceutical agents: Jun. 11, 1996, U.S. Pat. No. 5,525,348). We know about a series of patents from Friedman that deal with the production of dental varnish (M. Friedman, D. Steinerg, A. Soskolne: Sustained-release pharmaceutical compositions, Jun. 11, 1991, U.S. Pat. No. 5,023,082; M. Friedman, A. Sintov: Liquid polymer composition and method of use, Nov. 3, 1992, U.S. Pat. No. 5,160,737; M. Friedman, A. Sintov: Dental varnish composition and method of use, Jul. 19, 1994, U.S. Pat. No. 5,330,746; M. Friedman, A. Sintov: Dental varnish composition and method of use, Jul. 15, 1997, U.S. Pat. No. 5,648,399; M. Friedman, A. Sintov: Dental varnish composition and method of use, Jun. 17, 1997, U.S. Pat. No. 5,639,795). These patents are nearly identical with regard to their content and contain quaternary ammonium salts as essential antimicrobial substances. The patents describe paints and polymer solutions for their production, which largely consist of the following components: a copolymer, consisting of methacrylic acid and methacrylic acid esters, with free carboxylic acid groups, a copolymer, consisting of methacrylic acid and methacrylic acid methyl ester, with free carboxylic acid groups, a copolymer, consisting of dimethyl aminoethyl acrylate and ethyl methacrylate, and a copolymer, consisting of methylacrylate and chlorotrimethyl ammonium ethyl methacrylate. The interesting aspect in U.S. Pat. No. 5,648,399 is that a reagent, which influences the release of the active ingredient, from the group of cross-linking reagents, the polysaccharides, lipids, polyhydroxy compounds, polycarboxylid acids, divalent cations, citric acids, sodium citrate, sodium docusate, proteins, polyoxyethylene sorbitane mono-oleate and amino acids is added to the polymer combination.

Bayston and Grove present an interesting suggestion on the production of antimicrobial medicinal products (R. Bayston, N. J. Grove: Antimicrobial device and method, Apr. 17, 1990, U.S. Pat. No. 4,917,686). In this patent, antibiotic substances are dissolved in a suitable organic solvent. This solution is then allowed to react on the polymer surfaces that are supposed to be modified. The polymer swells due to the solvent, and the active ingredient can penetrate into the surface. Darouiche and Raad suggest basically the same method for the antimicrobial impregnation of catheters and other medical implants, where also an antimicrobial active ingredient is dissolved in an organic solvent (R. Darouiche, I. Raad: Antimicrobial impregnated catheters and other medical implants and method for impregnating catheters and other medical implants with an antimicrobial agent, Apr. 29, 1997, U.S. Pat. No. 5,624,704). This solution is allowed to react on the surface that is supposed to be treated, wherein the active ingredient penetrates into the material and is deposited there.

A method for coating surfaces with cationic antibiotics described by Lee represents an alternative to the systems described so far (C. C. Lee: Coating medical devices with cationic antibiotics, Jan. 23, 1990, U.S. Pat. No. 4,895,566). With this method, first a negatively charged heparin layer is applied onto the surface that is supposed to be coated and upon its adhesion this cationic antibiotic is allowed to be deposited. A similar solution is suggested by Greco et al, where first a solution of anionic surface-active substances is allowed to react on the surface that is to be coated (R. S. Greco, R. A. Harvey, S. Z. Trooskin: Drug bonded prosthesis and process for producing same, Nov. 7, 1989, U.S. Pat. No. 4,879,135). In this process, the anionic molecules adsorb on the surface. Subsequently cationic active ingredients, such as gentamicin, are electrostatically bound. With regard to the last two quoted methods, it should be noted that the charge density with antibiotics per surface unit is very limited, and that the adhesion of these coatings should be regarded with a critical eye.

Underlying the present invention is the objective of developing a flexible antibiotic(s)-polymer combination, which under physiological conditions permits a continuous release of antibiotics over a time period of several days to weeks and can be used both in human and veterinary medicine. This antibiotic(s)-polymer combination should be able to be applied to the surfaces of medical plastic and metal implants in a simple, yet adhesive manner. It is particularly important that the coating is flexible and elastic and that no toxic components are released. Furthermore, the flexible antibiotic(s)-polymer combination should be suitable for the production of antibiotic threads, foils and molded bodies.

The invention is based on the surprising finding that homogeneous polymer mixtures, consisting of one or more hydrophobic polymers from the groups of poly(methacrylic acid esters), the poly(acrylic acid esters), the poly(methacrylic acid ester-co-acrylic acid esters) and one or more hydrophilic polymers from the group of polyethers, in which one or more slightly water-soluble antibiotics from the groups of aminoglycoside antibiotics, the lincosamide antibiotics, the tetracycline antibiotics and quinolone antibiotics are suspended, form stable composites, which in an aqueous environment exhibit a release over a period of days. The subsequent explanation is a descriptive interpretation of presumably occurring processes. Upon introducing the composites in the aqueous environment, the hydrophilic polyether dissolves, wherein the hydrophobic, water-insoluble polymers remain as residue. This way microporous, interconnecting cavities are created in the remaining hydrophobic polymer matrix. This means that the formation of microporous, interconnecting cavities takes place only with the effect of an aqueous and/or physiological environment under in situ conditions. The slightly water-soluble antibiotics particles are physically encapsulated in this remaining hydrophobic polymer matrix. Due to the cavities formed this way, the aqueous environment can reach the slightly water-soluble antibiotics only upon the creation of these cavities. The release of antibiotics thus does not commence until during or after leaching out of the polyethers.

These hydrophilic polymers are toxicologically safe, and some of their representatives are described in European pharmacopoeia. The particular benefit of this antibiotic(s)-polymer combination consists of the fact that the antibiotics suspended in the homogeneous polymer mixture are protected from chemical and mechanical influences, such as abrasion, before being introduced into an aqueous, physiological environment. It is only through the in situ formation of the microporous, interconnecting cavities that the antibiotic(s)-polymer combination is opened up for the release of the antibiotics. By using slightly water-soluble antibiotics, they are leached out of the interconnecting cavities only slowly. Beyond that, it was surprisingly shown that the percentage of hydrophilic polyethers in the homogeneous polymer mixture can influence the release speed of the antibiotics.

The objective of the invention is accomplished in that, in a homogeneous polymer mixture, which consists of one or more hydrophobic polymers from the groups of poly(methacrylic acid esters), the poly(acrylic acid esters) and the poly(methacrylic acid ester-co-acrylic acid esters) and of one or more hydrophilic polymers from the group of polyethers, one or more slightly water-soluble antibiotics from the groups of aminoglycoside antibiotics, lincosamide antibiotics, tetracycline antibiotics, quinolone antibiotics, possibly in an easily water-soluble antibiotic from the groups of aminoglycoside antibiotics, lincosamide antibiotics, β-lactam antibiotics and tetracycline antibiotics and possibly one or more organic adjuvants are suspended, and that this suspension forms a composite.

The following embodiments have proven worthwhile in practice.

It is in accordance with the invention that the composite is formed through vaporization of propan-2-one and/or butan-2-one by a flowable suspension, which consists of a homogeneous mixture of propan-2-one and/or butan-2-one, one or more hydrophobic polymers from the groups of poly(methacrylic acid esters), poly(acrylic acid esters) and poly(methacrylic acid ester-co-acrylic acid esters) and one or more hydrophilic polymers from the group of polyethers, in which one or more slightly water soluble antibiotics from the groups of aminoglycoside antibiotics, lincosamide antibiotics, tetracycline antibiotics and quinolone antibiotics, possibly an easily water-soluble antibiotic from the groups of aminoglycoside antibiotics, lincosamide antibiotics, β-lactam antibiotics and tetracycline antibiotics, and possibly one or more organic adjuvants are suspended.

According to the invention, the composite is formed on the basis of a molten mass, which consists of one or more hydrophobic polymers from the groups of poly(methacrylic acid esters), poly(acrylic acid esters) and poly(methacrylic acid ester-co-acrylic acid esters) and one or more hydrophilic polymers from the group of polyethers, in which one or more slightly water soluble antibiotics from the groups of aminoglycoside antibiotics, lincosamide antibiotics, tetracycline antibiotics and quinolone antibiotics, possibly an easily water-soluble antibiotic from the groups of aminoglycoside antibiotics, lincosamide antibiotics and tetracycline antibiotics, and possibly one or more organic adjuvants are suspended.

Furthermore it is in accordance with the invention that the content of hydrophilic polymer in the homogeneous polymer mixture is between 0.1 and 60 mass percent.

According to the invention polyethylene glycol with a mean molar mass in the range of 120 gmol⁻¹ to 35,000 gmol⁻¹ is preferred as the polyether.

Also according to the invention polypropylene glycol with a mean molar mass in the range of 200 gmol⁻¹ to 35,000 gmol⁻¹ is preferred as the polyether.

According to the invention polyethylene glycol with a mean molar mass in the range of 200 gmol⁻¹ to 600 gmol⁻¹ is particularly preferred as the polyether.

According to the invention poly(methacrylic acid methyl esters), poly(methacrylic acid ethyl esters), poly(methacrylic acid propyl esters), poly(methacrylic acid-n-butyl esters), poly(methacrylic acid-n-hexyl esters), poly(methacrylic acid cyclohexyl esters), poly(acrylic acid methyl esters), poly(acrylic acid ethyl esters), poly(acrylic acid propyl esters), poly(acrylic acid butyl esters) and poly(acrylic acid cyclohexyl esters) with mean molar masses in the range of 20,000 gmol⁻¹ to 1,000,000 gmol⁻¹ are preferred as hydrophobic polymers.

Also according to the invention, copolymers and terpolymers with mean molar masses in the 20,000 gmol⁻¹ to 1,000,000 gmol⁻¹ range are preferred as hydrophobic polymers, which are produced from acrylic acid methyl ester, acrylic acid ethyl ester, acrylic acid propyl ester, acrylic acid-n-hexyl ester, acrylic acid cyclohexyl ester, methacrylic acid methyl ester, methacrylic acid ethyl ester, methacrylic acid propyl ester, methacrylic acid butyl ester, methacrylic acid-n-hexyl ester and methacrylic acid cyclohexyl ester.

According to the invention, sulfonamides and/or anti-inflammatory agents and/or anesthetics and/or vancomycin are preferred as organic adjuvants.

According to the invention, the flowable suspension forms composites in the shape of threads through a spinning process, while vaporizing propan-2-one and/or butan-2-one.

According to the invention, the flowable suspension forms composites in the shape of foils through a casting process, while vaporizing propan-2-one and/or butan-2-one.

According to the invention, the flowable suspension forms composites in the shape of powders and granules through an atomizing process, while vaporizing propane-2-one and/or butan-2-one.

According to the invention, the composite is formed into molded bodies and foils through pressing, extruding and rolling processes.

According to the invention, the polymer tubes, polymer threads, polymer foils, spherical polymer bodies, cylindrical polymer bodies and chain-shaped polymer bodies that are coated with the composite are used as medical implants.

According to the invention, catheters, tracheal cannulas and tubes for intraperitoneal nutrition are coated with the composite.

According to the invention, implantable metal plates, metal nails and metal screws are coated with the composite.

Furthermore it is in accordance with the invention that the composite is used for gluing together polymer bodies, polymer foils, polymer threads, metal plates and metal tubes for medical usage.

According to the invention, the composite is used as a binding agent for the production of antibiotic molded bodies from polymer granules, polymer powders, resorbable glass powders, non-resorbable glass powders and quartz powders.

According to the invention, the flowable suspension is applied through immersion, spraying, painting, brushing and rolling processes onto the surface of polymers and/or metals, and a composite in the form of a coating is formed by vaporizing propan-2-one and/or butan-2-one.

According to the invention, the composite is applied as a coating on polymer threads, polymer foils, polymer tubes, polymer bags and polymer bottles for medical usage.

According to the invention, the composite is applied as a coating onto spherical molded bodies, onto cylindrical molded bodies and onto chain-shaped molded bodies that consist of polymers and/or metal.

Furthermore it is in accordance with the invention that the composite is applied as a coating onto molded bodies, foils and strings made of poly(methacrylic acid ester), poly(acrylic acid ester), poly(methacrylic acid ester-co-acrylic acid ester), polyvinyl chloride, polyvinylidene chloride, silicone, polystyrene and polycarbonate.

It is also in accordance with the invention that the composite is used as a binding agent for the production of antibiotic laminates.

Furthermore it is in accordance with the invention that the composite is applied as a coating onto the surface of metals and/or polymers through a sintering process.

The invention will be explained in more detail with three examples:

### EXAMPLE 1

A solution consisting of 1.5 g poly(methyl methacrylate), 120 g polyethylene glycol 600 and 5 ml acetone is prepared. In this solution, 300 mg fine powdery gentamicin pentakis hexadecyl sulfonate and 300 mg gentamycin sulfate are suspended. This suspension is cast onto a glass plate. The acetone is allowed to become concentrated through evaporation. This creates a semi-transparent, elastic foil, which can be pulled off the glass plate.

### EXAMPLE 2

A solution consisting of 1.5 g poly(methyl methacrylate), 120 g polyethylene glycol 600 and 5 ml acetone is prepared. In this solution, 300 mg fine powdery gentamicin pentakis dodecyl sulfate and 300 mg gentamycin sulfate are suspended. Into this suspension, a 3 cm long piece of polyvinyl chloride tube (tube diameter 4 mm) is immersed. Subsequently, the coated polyvinyl chloride tube is allowed to dry at room temperature. This way an elastic adhesive coating on the polyvinyl chloride tube is obtained.

### EXAMPLE 3

Into a molten mass (150° C.), consisting of 2 g poly(methacrylic acid-co-acrylic acid methyl ester) and 200 g polyethylene glycol 600, 200 mg fine powdery gentamicin pentakis dodecyl sulfate are introduced and distributed evenly. Upon cooling of the molten material, a milky-cloudy solid composite is obtained.

1-27. (canceled)

28. An antibiotic(s)-polymer combination comprising:
a) a homogeneous polymer mixture comprising:
i) one or more hydrophobic polymers selected from the group consisting of poly(methacrylic acid esters), poly(acrylic acid esters) and poly(methacrylic acid ester-co-acrylic acid esters); and
ii) one or more hydrophilic polymers selected from the group consisting of polyethers;
b) at least one antibiotic which is slightly water-soluble and is selected from the group consisting of slightly water-soluble aminoglycoside antibiotics, slightly water-soluble lincosamide antibiotics, slightly water-soluble tetracycline antibiotics and slightly water-soluble quinolone antibiotics;
c) at least one antibiotic which is easily water-soluble and is selected from the group consisting of easily water-soluble aminoglycoside antibiotics, easily water-soluble lincosamide antibiotics, easily water-soluble β-lactam antibiotics and easily water-soluble tetracycline antibiotics; and
d) optionally one or more organic adjuvants;
wherein said combination is in the form of a suspension, or in the form of a composite obtained from said suspension.

29. Antibiotic(s)-polymer combination in accordance with claim 28, wherein the composite is formed from a vaporization of propan-2-one and/or butan-2-one of a flowable suspension, the flowable suspension comprising a homogeneous mixture of propan-2-one and/or butan-2-one, one or more hydrophobic polymers selected from the groups consisting of poly(methacrylic acid esters), poly(acrylic acid esters) and poly(methacrylic acid ester-co-acrylic acid esters) and one or more hydrophilic polymers selected from the group consisting of polyethers, in which
(a) an antibiotic which is slightly water soluble is selected from the groups consisting of slightly water soluble aminoglycoside antibiotics, slightly water soluble lincosamide antibiotics, slightly water soluble tetracycline antibiotics and slightly water soluble quinolone antibiotics;
(b) an antibiotic which is easily water-soluble selected from the groups consisting of easily water-soluble aminoglycoside antibiotics, easily water-soluble lincosamide antibiotics, easily water-soluble β-lactam antibiotics and easily water-soluble tetracycline antibiotics; and
(c) optionally one or more organic adjuvants are suspended therein.

30. Antibiotic(s)-polymer combination in accordance with claim 28, wherein the composite is formed from a molten mass, which comprises one or more hydrophobic polymers selected from the groups consisting of poly(methacrylic acid esters), poly(acrylic acid esters) and poly(methacrylic acid ester-co-acrylic acid esters) and one or more hydrophilic polymers selected from the group of polyethers, in which
a) the antibiotic which is slightly water soluble is selected from the groups consisting of slightly water soluble aminoglycoside antibiotics, slightly water soluble lincosamide antibiotics, slightly water soluble tetracycline antibiotics and slightly water soluble quinolone antibiotics;
(b) the antibiotic which is easily water-soluble is selected from the groups consisting of easily water-soluble aminoglycoside antibiotics, easily water-soluble lincosamide antibiotics and easily water-soluble tetracycline antibiotics; and
(c) optionally one or more organic adjuvants are suspended therein.

31. Antibiotic(s)-polymer combination in accordance with claim 28, wherein the hydrophilic polymer in the homogeneous polymer mixture is between 0.1 to 60 percent by mass.

32. Antibiotic(s)-polymer combination in accordance with claim 28, wherein the polyether is a polyethylene glycol with a mean molar mass in the range of 120 gmol⁻¹ to 35,000 gmol⁻¹.

33. Antibiotic(s)-polymer combination in accordance with claim 28, wherein the one or more hydrophobic polymers are selected from the group consisting of poly(methacrylic acid methyl esters), poly(methacrylic acid ethyl esters), poly(methacrylic acid propyl esters), poly(methacrylic acid-n-butyl esters), poly(methacrylic acid-n-hexyl esters), poly(methacrylic acid cyclohexyl esters), poly(acrylic acid methyl esters), poly(acrylic acid ethyl esters), poly(acrylic acid propyl esters), poly(acrylic acid butyl esters) and poly(acrylic acid cyclohexyl esters) each of which has a mean molar mass in the range of 20,000 gmol⁻¹ to 1,000,000 gmol⁻¹.

34. Antibiotic(s)-polymer combination in accordance with claim 28, wherein the one or more hydrophobic polymers are selected from copolymers and terpolymers with mean molar masses in the range of 20,000 gmol⁻¹ to 1,000,000 gmol⁻¹, which copolymers and terpolymers are produced from at least one polymer selected from the group consisting of acrylic acid methyl ester, acrylic acid ethyl ester, acrylic acid propyl ester, acrylic acid-n-hexyl ester, acrylic acid cyclohexyl ester, methacrylic acid methyl ester, methacrylic acid ethyl ester, methacrylic acid propyl ester, methacrylic acid butyl ester, methacrylic acid-n-hexyl ester and methacrylic acid cyclohexyl ester.

35. Antibiotic(s)-polymer combination in accordance with claim 28, wherein the organic adjuvants are one or more members selected from the group consisting of sulfonamides, anti-inflammatory agents, and anesthetics.

36. Antibiotic(s)-polymer combination in accordance with claim 29, wherein the flowable suspension forms composites in the shape of threads through a spinning process, while vaporizing propan-2-one and/or butan-2-one.

37. Antibiotic(s)-polymer combination in accordance with claim 29, wherein the flowable suspension forms composites in the shape of foils through a casting process, while vaporizing propan-2-one and/or butan-2-one.

38. Antibiotic(s)-polymer combination in accordance with claim 29, wherein the flowable suspension forms composites in the shape of powders and granules through an atomizing process, while vaporizing propan-2-one and/or butan-2-one.

39. Antibiotic(s)-polymer combination in accordance with claim 28, which is a composite formed into molded bodies and foils through pressing, extruding and rolling processes.

40. An implant comprising an antibiotic(s)-polymer combination according to claim 28.

41. The implant according to claim 40, which is in the form of one or more of polymer tubes, polymer threads, polymer foils, spherical polymer bodies, cylindrical polymer bodies and chain-shaped polymer bodies that are coated with the antibiotic(s)-polymer combination.

42. A catheter, tracheal cannulas or tube for intraperitoneal nutrition which is coated with an antibiotic(s)-polymer combination according to claim 28.

43. An implantable metal plate, a metal nail or a metal screw which is coated with an antibiotic(s)-polymer combination according to claim 28.

44. A construct comprising one or more of polymer bodies, polymer foils, polymer threads, metal plates and metal tubes held together by an antibiotic(s)-polymer combination according to claim 28.

45. An antibiotic molded body comprising one or more of polymer granules, polymer powders, resorbable glass powders, non-resorbable glass powders and quartz powders held together by an antibiotic(s)-polymer combination according to claim 28.

46. A process of forming an antibiotic material comprising
a) providing an antibiotic(s)-polymer combination according to claim 28, said antibiotic(s)-polymer combination being in the form of a flowable suspension;
b) applying the flowable suspension onto a surface of at least one of polymers and/or metals by at least one of the processes selected from the group consisting of immersion, spraying, painting, brushing and rolling, and
c) forming a composite in the form of a coating by vaporizing propane-2-one and/or butane-2-one.

47. A process of forming an antibiotic material comprising providing an antibiotic(s)-polymer combination according to claim 28, and applying said antibiotic(s)-polymer combination as a coating on polymer threads, polymer foils, polymer tubes, polymer bags and polymer bottles.

48. A process of forming an antibiotic material comprising providing an antibiotic(s)-polymer combination according to claim 28, and applying said antibiotic(s)-polymer combination as a coating on at least one of spherical molded bodies, cylindrical molded bodies and chain-shaped molded bodies that comprise polymer and/or metal.

49. Antibiotic(s)-polymer combination in accordance with claim 28, wherein the slightly water-soluble antibiotic is a slightly water-soluble form of gentamicin and the easily water-soluble antibiotic is an easily water-soluble form of gentamicin.

50. Antibiotic(s)-polymer combination comprising a homogeneous polymer mixture and gentamicin, wherein the homogeneous polymer mixture consists of polymethylmethacrylate and polyethylene glycol.
